(19) United States
(12) Patent Application Publication
(10) Pub. No.: US 2012/0302952 A1
(43) Pub. Date: Nov. 29, 2012

(54) CATHETER

(75) Inventors: Noriyuki KITADA, Shizuoka (JP); Yuichi TADA, Kanagawa (JP)

(73) Assignee: Terumo Kabushiki Kaisha, Shibuya-ku (JP)

(21) Appl. No.: 13/568,751

(22) Filed: Aug. 7, 2012

Related U.S. Application Data
(63) Continuation of application No. PCT/JP2011/054706, filed on Mar. 2, 2011.

(30) Foreign Application Priority Data
Mar. 5, 2010 (JP) 2010-049541

(51) Int. Cl. A61M 25/14; A61M 25/10

(52) U.S. Cl. 604/96.01; 604/523; 604/525

(57) ABSTRACT

A catheter includes an outer tube, and an inner tube which is disposed inside the outer tube and through which a guide wire is passed via a distal-side opening and a proximal-side opening. The outer tube has, in the axial direction thereof, at least: a first region on the distal side; a second region disposed on the proximal side and higher in rigidity than the first region; and a transition region which is provided between the first region and the second region and of which rigidity varies from the same rigidity as that of the first rigidity to the same rigidity as that of the second rigidity. The outer tube is configured with an opening, to which the proximal-side opening of the inner tube is connected, being provided in the second region.

### CROSS-REFERENCES TO RELATED APPLICATIONS

This application is a continuation of International Application PCT/JP2011/054706 filed on Mar. 2, 2011, which claims priority to Japanese Patent Application No. 2010-049541 filed in the Japanese Patent Office on Mar. 5, 2010, the entire content of both of which is incorporated herein by reference.

### TECHNICAL FIELD

The present invention generally relates to a catheter in which an outer tube is provided at an intermediate portion of the catheter with an opening through which a guide wire is led out.

### BACKGROUND DISCUSSION

An example of a treatment of cardiac infarction or stenocardia involves a method in which a lesion part (stenosed part) of a coronary artery is dilated by a balloon mounted on a distal end of a catheter. A similar method may also be practiced for improving a stenosed part (narrow section) formed in other biorgans such as other blood vessels, bilary duct, trachea, esophagus, urethra, and other organs. Such a catheter has a long shaft main body, and a guide wire precedently introduced into a living body is passed through the shaft main body, whereby the catheter can be advanced along the guide wire into the living body.

Japanese Patent Laid-open No. 2000-217923 describes a balloon catheter which includes an inner tube shaft formed with a wire lumen for passing a guide wire therethrough, and an outer tube shaft disposed on the outer circumference side of the inner tube shaft, and in which a balloon is provided at a distal portion. This balloon catheter adopts a structural system ordinarily called “rapid exchange type” in which the outer tube shaft composed of a single tube is provided at an intermediate portion thereof with an opening, and a proximal portion of the inner tube shaft is joined to the opening so as to form a guide wire leading-out port.

In general, when a proximal portion of a catheter is operated by an operator, a long shaft must thereby be smoothly advanced through a bent blood vessel. In addition, the distal end of the shaft (catheter) must smoothly penetrate a hard stenosed part. Accordingly, it is desirable for a pushing-in force exerted by the operator from the proximal side to be transmitted assuredly to the distal side.

In a configuration described in the Japanese Patent Laid-open No. 2000-217923, the outer tube shaft composed of a single tube and having a predetermined rigidity is formed with an opening at an intermediate portion thereof, and a proximal portion of the inner tube shaft is joined to the opening so as to form a guide wire leading-out port. Therefore, the pushing-in force exerted from the proximal side would be largely absorbed in the opening part constituting a rigidity change point, so that the pushing-in force may fail to be sufficiently transmitted to the distal side.

On the other hand, a structure has also been devised in which an outer tube shaft is composed of two members consisting of a flexible distal-side shaft and a highly rigid proximal-side shaft and an opening is provided at a joint part between the two shafts. In the case of this structure, however, a stress relevant to a load such as a tensile load or a bending load is concentrated in the vicinity of the joint part, so that the opening may become a starting point of kinking or breakage and the pushing-in force transmission performance may be lowered.

### SUMMARY

According to one aspect, a catheter comprises an outer tube possessing a distal end portion terminating at a distal-most end and a proximal end portion terminating at a proximal-most end, with the outer tube including a tube wall surrounding an interior of the outer tube; an inner tube disposed within the interior of the outer tube so that the outer tube surrounds and axially overlaps a proximal portion of the inner tube, and wherein the inner tube possesses a distal end extending distally beyond the distal-most end of the outer tube, and wherein the inner tube includes a wire lumen extending along a longitudinal extent of the inner tube between a distal-end opening at the distal end of the inner tube and a proximal-end opening at a proximal end of the inner tube, with the wire lumen being configured to receive a guide wire passing through both the distal-end opening and the proximal-end opening. The outer tube includes a through opening passing through the wall of the outer tube and opening to the interior of the outer tube, with the opening being located distal of the proximal-most end of the outer tube, and wherein the proximal-most end of the inner tube is fixed in the opening. The outer tube includes a first region, a second region and a transition region arranged along an axial extent of the outer tube, with the transition region located axially between the first region and the second region, the first region positioned distally of the transition region, and the second region being positioned proximally of the transition region. The second region possesses a rigidity greater than the rigidity of the first region, and the transition region between the first region and the second region possesses a rigidity which gradually varies from a distal end of the transition region which possesses the same rigidity as the rigidity of the first region to a proximal end of the transition region which possesses the same rigidity as the rigidity of the second region. The opening in the outer tube is located in the second region of the outer tube whose rigidity is greater than the rigidity of the first region.

According to another aspect, a catheter includes: an outer tube; and an inner tube which is disposed within the outer tube and through which a guide wire is passed via a distal-side opening and a proximal-side opening, wherein the outer tube includes, in an axial direction thereof, at least a first region on a distal side, a second region which is on a proximal side and which is higher in rigidity than the first region, and a transition region which is provided between the first region and the second region and which varies in rigidity from the same rigidity as the rigidity of the first region to the same rigidity as the rigidity of the second region; and the outer tube is provided in the second region thereof with an opening to which the proximal-side opening of the inner tube is connected.

The opening for leading out the guide wire is thus provided at an intermediate portion of an outer tube which includes a flexible first region, a relatively highly rigid second region and a transition region which varies in rigidity between the first region and the second region, and the opening portion is formed in the second region which is relatively high in rigidity. In other words, the outer tube is provided with the transition region which varies in rigidity from a low rigidity on the distal side to a high rigidity on the proximal side, and the opening is formed in the region the highest in rigidity or the region high to some extent in rigidity, of a plurality of regions differing in rigidity. This makes it possible to minimize the absorption in the opening part of the pushing-in force transmitted from the proximal side, and to maintain at a quite high value the coefficient of transmission of the pushing-in force from the proximal side to the distal side of the catheter. In addition, since the catheter is so configured that shaft rigidity is gradually lowered (made more flexible) from the proximal side toward the distal side, the catheter can be smoothly advanced through a bent blood vessel or into a stenosed part having a rugged shape.

With the first region and the second region formed respectively from resins differing in rigidity, and the transition region formed so that the mixing ratio of the resin of the first region and the resin of the second region gradually varies in the axial direction, the outer tube is configured as an integrally molded, unitary, one-piece tube so that no joint part is formed at any intermediate portion of this tube and, in addition, the rigidity of the outer tube can be varied more smoothly, making it possible to eliminate a region where rigidity varies abruptly. Accordingly, it is possible to effectively obviate a situation in which the joint part or a rigidity change point would become a starting point of kinking or breakage in the presence of a load such as a tensile load or a bending load.

Where the first region and the second region and the transition region are integrally molded by extrusion by use of a resin change-over die, an outer tube varying in rigidity more smoothly can be easily molded.

With the catheter configured as a balloon catheter including a balloon which is attached on its proximal side to a distal portion of the outer tube and which is attached on its distal side to a distal portion of the inner tube, the balloon can be rather easily advanced to a stenosed part in a living body. The balloon can also be quite assuredly disposed with a sufficient pushing-in force, even in a hard stenosed part or the like.

Even if the catheter is configured so that an opening for leading out a guide wire is provided at an intermediate portion of an outer tube, a configuration in which a flexible first region and a highly rigid second region and a transition region provided between the first and second regions and varying in rigidity are provided and in which the opening is formed in the second region where the rigidity is high makes it possible to minimize the absorption in the opening part of the pushing-in force exerted from the proximal side, and to maintain at a high value the coefficient of transmission of the pushing-in force from the proximal side to the distal side of the catheter. Moreover, since the catheter is so configured that shaft rigidity is gradually lowered (made more flexible) from the proximal side toward the distal side, the catheter can be smoothly advanced through a bent blood vessel or into a stenosed part having a rugged shape.

### BRIEF DESCRIPTION OF THE DRAWINGS

FIG. 1 shows a general configuration of a catheter according to a first embodiment serving as an example of the catheter disclosed here.

FIG. 2A is a plan view showing, in an enlarged form, the distal side of the catheter shown in FIG. 1, and FIG. 2B is a longitudinal cross-sectional side view of the catheter shown in FIG. 2A.

FIG. 3A is a plan view of an outer tube model modeled after an outer tube, and FIG. 3B is a graph showing the relationship between axial-directional position and resisting load in the outer tube model shown in FIG. 3A.

FIG. 4 is a block diagram of a manufacturing apparatus for carrying out an example of a method of manufacturing an outer tube.

FIG. 5 shows a configuration of a measuring apparatus for measuring the coefficient of transmission of a pushing-in load in the axial direction of the outer tube.

FIG. 6 is a graph showing the relationship between pushing-in load and coefficient of transmission of load, at each position of an opening formed in the outer tube model.

FIG. 7A is a partly omitted plan view of an outer tube according to a modification, and FIG. 7B is a graph showing the relationship between axial-directional position and resisting load in the outer tube shown in FIG. 7A.

FIG. 8 shows a general configuration of a modified version of the catheter.

FIG. 9 is an enlarged view of the distal side of the catheter shown in FIG. 8.

### DETAILED DESCRIPTION

The catheter 10 according to the present embodiment is a so-called PTCA (Percutaneous Transluminal Coronary Angioplasty) dilation catheter in which an elongated shaft main body 12 is inserted into a biorgan, for example, a coronary artery, and a balloon 14 provided at a distal portion of the shaft main body 12 is outwardly expanded in a stenosed part (lesion part) to dilate the stenosed part for treatment of the stenosed part. The invention here is applicable also to a catheter for treatment of a lesion part of other biorgan such as other blood vessels, bile duct, trachea, esophagus, urethra and other organs, for example, a self-expandable stent catheter.

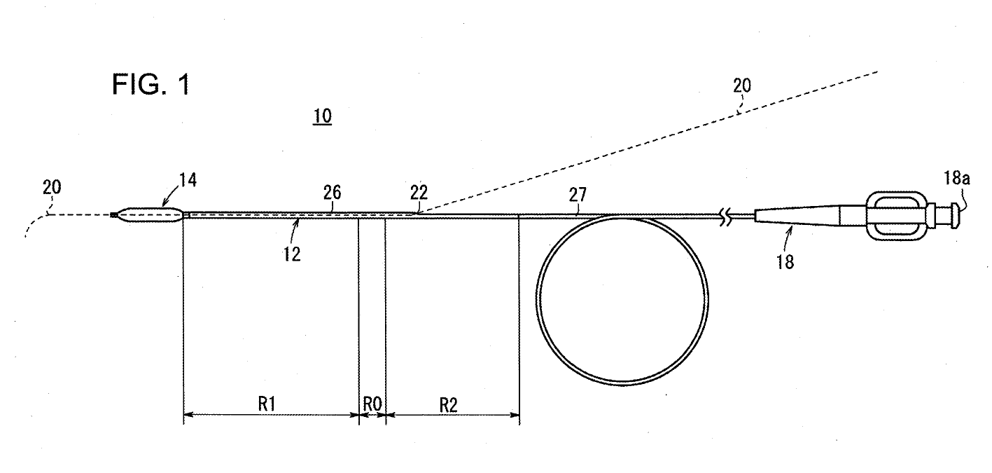

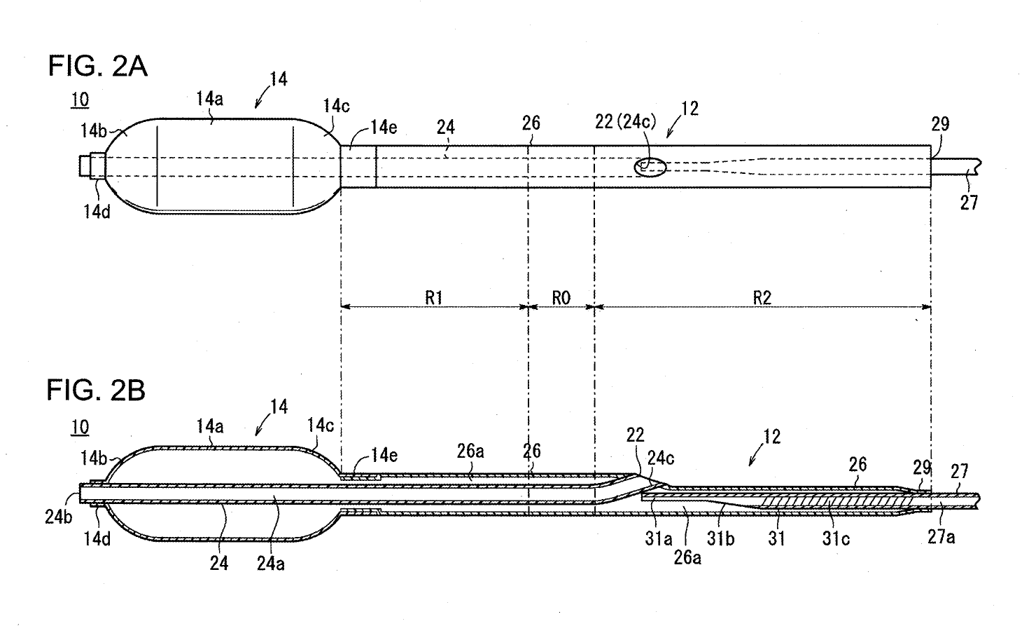

As shown in FIG. 1, the catheter 10 includes the elongated (long) shaft main body 12, the balloon 14 provided at a distal portion of the shaft main body 12, and a hub 18 provided at a proximal portion of the shaft main body 12. The catheter 10 is a so-called rapid exchange type catheter, wherein an opening 22 for leading out a guide wire 20 therethrough is provided at a position slightly on the distal side of a middle portion of the shaft main body 12. In FIGS. 1 and 2, the right side (the hub 18 side) of the shaft main body 12 is referred to the “proximal” side or end, and the left side (the balloon 14 side) of the shaft main body 12 is referred to as the “distal” side or end. This also applies to the other drawing figures as well.

As shown in FIGS. 2A and 2B, the shaft main body 12 includes an inner tube (inner tube shaft, or guide wire tube) 24 in which is provided a wire lumen 24a for passing a guide wire 20 therethrough, an outer tube (outer tube shaft, or distal shaft) 26 which defines between itself (inner circumferential surface of the outer tube 26) and the outer circumferential surface of the inner tube 24 an expansion lumen 26a for supplying an expanding (inflating) fluid for the balloon 14, and a proximal shaft 27 of which a distal portion is positioned in and joined to a proximal portion of the outer tube 26. The portion of the shaft main body 12 ranging from the distal end to the opening 22 is thus configured as a concentric double-walled tube.

The inner tube 24 extends through the inside of the balloon 14 and the outer tube 26, and is configured so that the vicinity of the distal end of the inner tube 24 is joined to a distal portion of the balloon 14 in a liquid-tight manner, and a proximal-side opening 24c opening at the proximal end of the inner tube 24 is joined or fixed to the opening 22 at an intermediate portion of the outer tube 26 in a liquid-tight manner by adhesion, heat fusing (welding) or the like. Therefore, the guide wire 20 inserted into the inner tube 24 via a distal-side opening 24b of the inner tube 24 serving as an entrance passes through the wire lumen 24a of the inner tube 24 from the distal end toward the proximal end, and is led out to the exterior via the opening 22 (the proximal-side opening 24c) serving as the exit.

The outer tube 26 extends from the proximal end of the balloon 14 to a joint part 29 between the outer tube 26 and the proximal shaft 27. The part of the outer tube 26 from the distal end to the opening 22 constitutes a double-walled tube where it defines the expansion lumen 26a between itself and the inner tube 24. Further, the part of the outer tube 26 from the opening 22 to the joint part 29 is a part in which a distal portion 31 of the proximal shaft 27 is positioned and which forms the expansion lumen 26a continuous with an expansion lumen 27a of the proximal shaft 27.

The proximal shaft 27 has the distal portion 31 formed in the shape of a trough inclined relative to the axial direction, by cutting a tube in a direction along the axial direction and in a direction inclined from the direction along the axial direction. The portion of the proximal shaft 27 on the proximal side of the distal portion 31 of the proximal shaft 27 is formed as a tube extending to the hub 18. The distal portion 31 has a slender distalmost portion 31a, and a slant portion 31b increasing in outer diameter in a slanted or inclined manner from the proximal side of the distalmost portion 31a. In addition, the distal portion 31 has a spiral slit 31c extending over a portion ranging from the proximal portion of the slant portion 31b to the joint part 29. This spiral slit 31 helps ensure that tube rigidity varies gradually. As a result, the distal portion 31 is so configured that its rigidity gradually increases from its distal end toward its proximal end.

The proximal shaft 27 and the outer tube 26 can feed into the balloon 14 an expanding (inflating) fluid fed under pressure from a pressurizing device such as an indeflator by, for example, a Luer taper 18a provided at the hub 18.

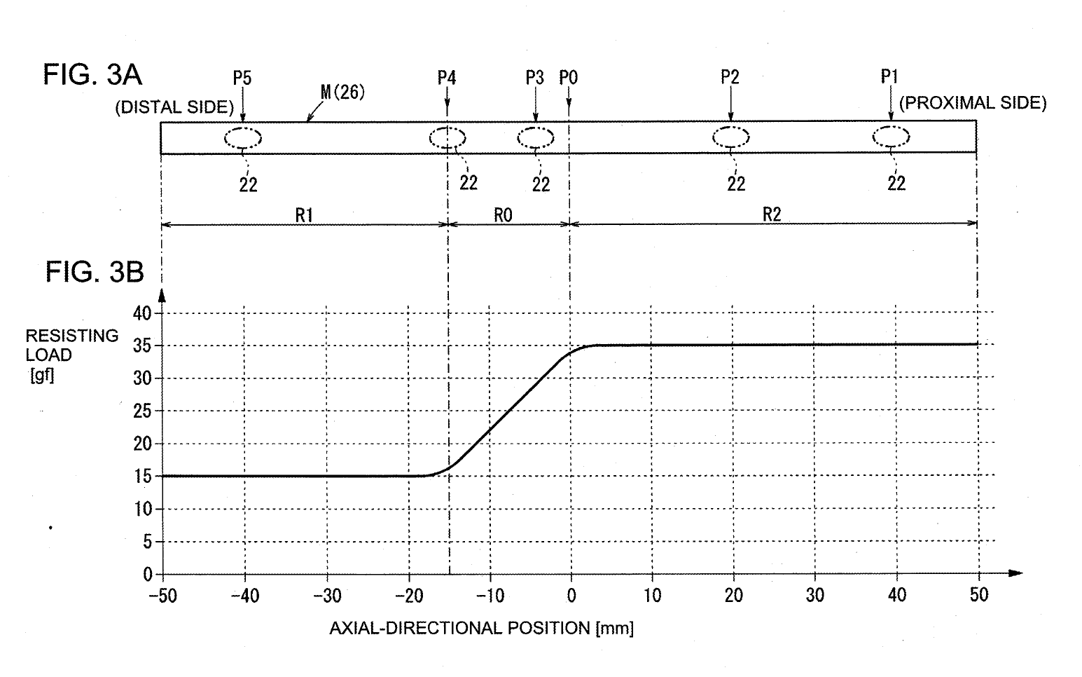

In the case of the present embodiment, the outer tube 26 is a tube which has a flexible first region R1 provided on the distal side and joined to the balloon 14, a second region R2 provided on the proximal side, higher in rigidity than the first region R1 and including a portion joined to the hub 18, and a transition region R0 provided between the first region R1 and the second region R2 which varies in rigidity to offer a continuation in rigidity between the first region R1 and the second region R2 (see the graph in FIG. 3B, as well). These regions are integrally molded in one piece in series with one another along the axial direction. The outer tube 26 is configured with the opening 22, to which the proximal-side opening 24c of the inner tube 24 is joined, being provided in the second region R2 which is on the proximal side relative to the transition region R0 and which has the highest or greatest rigidity (see FIGS. 2A and 2B).

The inner tube 24 is, for example, a tube which has an outside diameter of about 0.1 to 1 mm, preferably about 0.3 to 0.7 mm, a wall thickness of about 10 to 150 μm, preferably about 20 to 100 μm, and a length of about 10 to 2,000 mm, preferably about 20 to 1,500 mm, and the outside diameter and the inside diameter may be different between the distal side and the proximal side. The outer tube 26 is, for example, a tube which has an outside diameter of about 0.3 to 3 mm, preferably about 0.5 to 1.5 mm, a wall thickness of about 10 to 150 μm, preferably about 20 to 100 μm, and a length of about 30 to 2,000 mm, preferably about 40 to 1,600 mm, and the outside diameter and the inside diameter may be different between the distal side and the proximal side. With respect to the outer tube 26, for example, the length of the first region R1 is about 10 to 500 mm, the length of the transition region R0 is about 10 to 500 mm, and the length of the second region R2 is about 10 to 1,500 mm. The proximal shaft 27 is, for example, a tube which has an outside diameter of about 0.5 to 1.5 mm, preferably about 0.6 to 1.3 mm, an inside diameter of about 0.3 to 1.4 mm, preferably about 0.5 to 1.2 mm, and a length of about 800 to 1,500 mm, preferably about 1,000 to 1,300 mm.

The inner tube 24, the outer tube 26 and the proximal shaft 27 desirably have an appropriate degree of flexibility and an approximate degree of strength (rigidity) so that the elongated shaft main body 12 can be relatively smoothly inserted into or passed through a biorgan such as a blood vessel while the operator grips and operates a proximal portion. In view of this, the inner tube 24 and the outer tube 26 are preferably formed from a polymeric material such as polyolefins (e.g., polyethylene, polypropylene, polybutene, ethylene-propylene copolymer, ethylene-vinyl acetate copolymer, ionomer, or a mixture of two or more of them), polyvinyl chloride, polyamides, polyamide elastomers, polyurethane, polyurethane elastomers, polyimides, fluororesins, or mixtures thereof, or be composed of a multilayer tube formed from two or more of the just-mentioned polymeric materials. On the other hand, the proximal shaft 27 is desirably formed from a material having a comparatively high rigidity, examples of which include Ni—Ti alloy, brass, stainless steel (SUS), and aluminum; naturally, resins such as polyimides, polyvinyl chloride or polycarbonate may also be used to form the proximal shaft 27.

In the case of the present embodiment, since the outer tube 26 includes the three regions (the first region R1, the second region R2, and the transition region R0) as described above, the first region R1 and the second region R2 are formed respectively from different materials (different compositions of materials), and the transition region R0 is formed by use of a material in which the mixing ratio of the material of the first region R1 and the material of the region R2 is varied along the axial direction. The outer tube 26 may, naturally, be formed in a different manner. For instance, a configuration may be adopted in which the outer tube 26 is entirely formed from the same material throughout all the regions but the wall thickness or the like is varied, whereby rigidity is varied structurally. Specifically, for example, a configuration may be adopted in which the first region R1 has a fixed small wall thickness, the second region R2 has a fixed large wall thickness, and the transition region R0 has a wall thickness which varies gradually.

The structure of the outer tube 26 will now be described specifically below by showing the results of an experiment conducted using an outer tube model M modeled after the outer tube 26.

FIG. 3A is a plan view of the outer tube model M modeled after the outer tube 26, and FIG. 3B is a graph showing the relationship between axial-directional position (mm) and resisting load (gf) of the outer tube model M shown in FIG. 3A. The outer tube model M shown in FIG. 3A is a tube having an overall length of 200 mm (shorter than the outer tube 26) and an outside diameter of 1 mm. In FIG. 3B, the axis of abscissa represents the axial-directional position (distance) (mm) along the length of the outer tube model, with the proximal end of the transition region R0 of the outer tube model M shown in FIG. 3A being taken as an origin, and the axis of ordinates represents the resisting load (gf) of the outer tube model M at the corresponding axial-directional position. The resisting load (gf) is measured as an indicator of the level of rigidity at each axial-directional position of the outer tube model M. Specifically, a measurement position of the outer tube model M was disposed at the midpoint of two-point support beams with the interval between two support points set to 9 mm, a pushing-in load in a direction orthogonal to the axial direction was exerted on the outer tube model M at the measurement position in a pushing-in distance of 0.2 mm (at a pushing-in rate of 5 mm/min), and the load resistance (gf) upon such exertion was measured.

First, in FIG. 3B, the solid-line graph indicates an example of the structure of the outer tube 26 (variations in rigidity at each portion). For example, the outer tube 26 can be provided with a configuration such that the resisting load in the flexible first region R1 is about 15 gf, the resisting load in the highly rigid second region R2 is about 35 gf, and the resisting load in the transition region R0 interconnecting the first and second regions varies in the range of 15 to 35 gf. The transition region R0 may have other configurations than the configuration in which the resisting load therein varies rectilinearly, or in a proportional manner. For example, a configuration may be adopted in which the rigidity varies stepwise. In short, it suffices for the transition region R0 to be so configured that rigidity does not vary abruptly between the first region R1 and the second region R2 located on the distal and proximal sides thereof, in other words, the mixing ratio of resins different in rigidity varies between 100:0 and 0:100 between the distal and proximal sides of the transition region R0 and, at the same time, the mixing ratio of the resins different in rigidity varies gradually in the transition region R0.

The data plotted with circles in FIG. 3B are the results of measurement of load resistance of the outer tube model M measured using the two-point support beams. The data on the outer tube model M, also, show that the resisting load varies gradually from the first region R1 through the transition region R0 to the second region R2, though some scattering of the measured values exists.

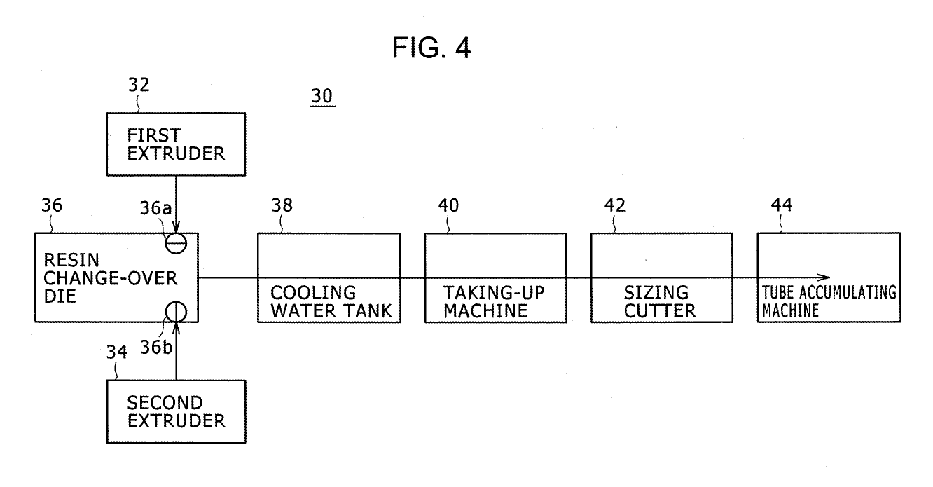

Now, an example of the method of manufacturing the outer tube 26 described above will be set forth. FIG. 4 is a block diagram of a manufacturing apparatus 30 for carrying out an example of the method of manufacturing the outer tube 26.

As shown in FIG. 4, the manufacturing apparatus 30 includes a first extruder 32 for extruding a predetermined resin A, a second extruder 34 for extruding another resin B higher in rigidity (for example, resisting load) than the resin A, and a resin change-over die 36 for performing kneading and molding while appropriately controlling the mixing ratio of the resins A and B extruded from the first extruder 32 and the second extruder 34. The resin change-over die 36 is provided with a change-over valve 36a configured to change the kneading ratio of the resin A fed from the first extruder 32 and a change-over valve 36b configured to change the kneading ratio of the resin B fed from the second extruder 34. Further, the manufacturing apparatus 30 includes a cooling water tank 38 for cooling a molded tube led out of the resin change-over die 36, a taking-up machine 40 for withdrawing the tube outputted from the resin change-over die 36, a sizing cutter 42 for cutting the molded long tube to a size corresponding to the outer tube 26, and a tube accumulating machine 44 for accumulating the molded and cut tubes.

Specifically, in the manufacturing apparatus 30, for example, pellets of the resin A for forming the flexible first region R1 of the outer tube 26 are charged into the first extrude 32, pellets of the resin B for forming the highly rigid second region R2 are charged into the second extruder 34, and the opening/closing timings of the change-over valves 36a and 36b are appropriately controlled, whereby the outer tube 26 having the different-rigidity regions molded integrally can be continuously manufactured as a single tube. Examples of the materials for the resin A and the region B include nylon elastomers; specific examples of the resin A include “PEBAX (registered trademark) No. 5533,” and specific examples of the resin B include “PEBAX (registered trademark) No. 7033.”

More specifically, in molding the first region R1, only the change-over valve 36a is set open whereas the change-over valve 36b is kept closed, whereby a tube is molded only from the resin A fed from the first extruder 32. Subsequently, in molding the transition region R0, starting from the condition where the change-over valve 36a is set open and the change-over valve 36b is kept closed, the opening amount of the change-over valve 36a is gradually reduced and, simultaneously, the opening amount of the change-over valve 36b is gradually increased, finally resulting in that only the change-over valve 36b is open whereas the change-over valve 36a is closed. By such operations, a tube is molded while the mixing ratio of the resin A fed from the first extruder 32 and the resin B fed from the second extruder 34 is varied from 100:0 to 80:20, then through 60:40 and 40:60 to 20:80 and eventually to 0:100. Finally, in molding the second region R2, only the change-over valve 36b is open whereas the change-over valve 36a is kept closed, whereby a tube is molded only from the resin B fed from the second extruder 34. The transition region R0 is thus configured to have a proximal half, possessing a greater amount of the resin (resin composition) B than the resin (resin composition) A, and a distal half, possessing a greater amount of the resin (resin composition) A than the resin (resin composition) B. The middle of the transition region R0 possesses equal amounts of resin (resin composition) A and resin (resin composition) B.

Thus, by using the manufacturing apparatus 30 in which the resin change-over die 36 is used, the outer tube 26 having a varying rigidity can be integrally molded as a single tube, while eliminating any joint part between adjacent ones of the regions and while eliminating any region where rigidity varies abruptly.

Meanwhile, in the outer tube 26, the opening 22 to which the proximal-side opening 24c of the inner tube 24 is to be joined is provided in the highly rigid second region R2 (see FIGS. 2A and 2B). The opening 22 influences the rigidity of the outer tube 26, as mentioned above.

In view of this, the disposition of the opening 22 in the outer tube 26 will be described specifically, by showing as an example the results of an experiment in which the disposition of the opening 22 in the outer tube model M shown in FIG. 3A is changed, and the coefficient of transmission of load from the proximal side to the distal side is measured.

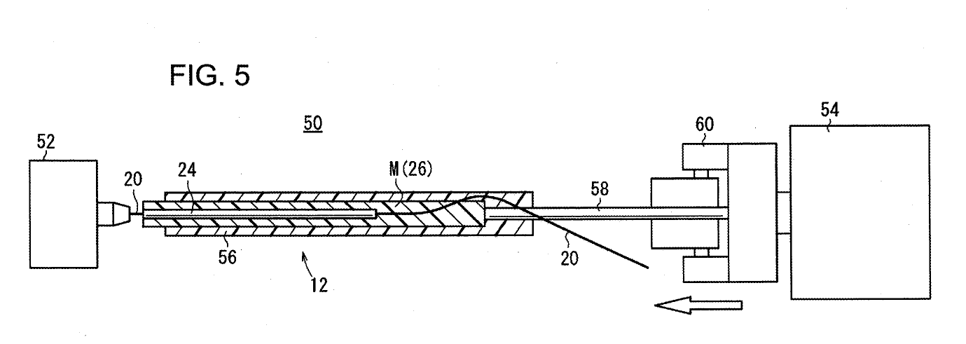

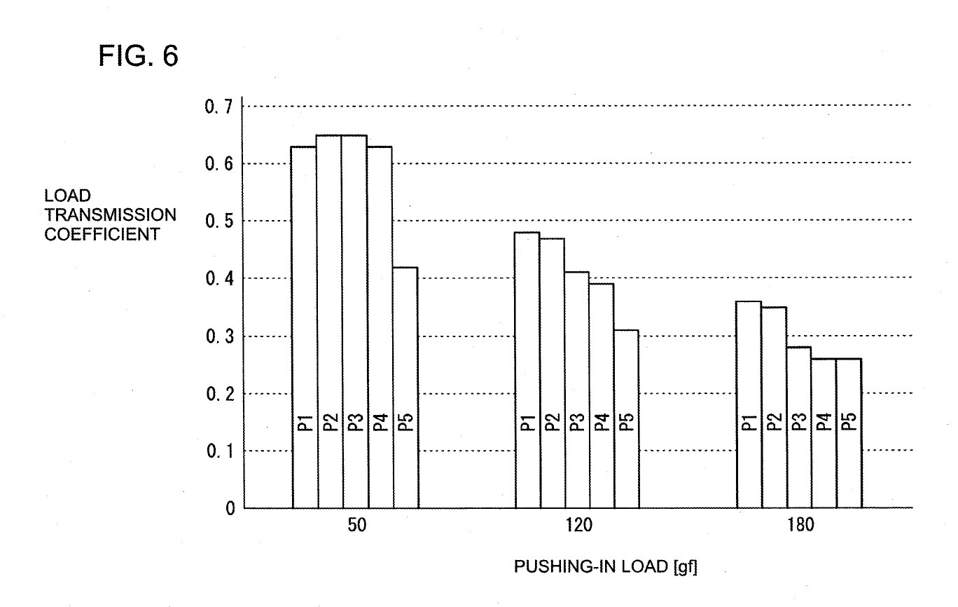

FIG. 5 shows the configuration of a measuring apparatus 50 for measuring the coefficient of transmission of a pushing-in load in the axial direction of the outer tube 26. In FIG. 5 is shown a configuration wherein the outer tube model M modeled after the outer tube 26 is disposed. FIG. 6 is a graph showing the relationship between pushing-in load (gf) and coefficient of transmission of load (a load transmission coefficient of 100% is taken as 1) at each position of the opening 22 formed in the outer tube model M. In FIG. 6, the axis of the abscissa represents a distal pushing-in load (gf) exerted on the proximal side of the outer tube model M, and the axis of the ordinate represents the coefficient of transmission of load at each position of the opening 22 under each pushing-in load. As indicated by two-dotted chain lines in FIG. 3A, the position of formation of the opening 22, in relation to the proximal end of the transition region R0 that is taken as an origin P0, was set at each of five points, namely, point P1 (40 mm to the proximal side from the origin P0), point P2 (20 mm to the proximal side from the origin P0), point P3 (5 mm to the distal side from the origin P0), point P4 (15 mm to the distal side from the origin P0), and point P5 (40 mm to the distal side from the origin P0). The pushing-in load was set at three levels, namely 50 gf, 120 gf, and 180 gf.

First, as shown in FIG. 5, the measuring apparatus 50 includes a first push-pull force gauge 52 and a second push-pull force gauge 54 for measuring a load on the distal side and the proximal side of an outer tube 26 (in this case, the outer tube model M), a silicone tube 56 for axially slidably supporting the outer tube 26 which is moved toward the distal side by receiving a load from the proximal side between the two push-pull force gauges 52 and 54, a proximal shaft 58 connected to the proximal side of the outer tube 26, and a clamp mechanism 60 for clamping the proximal side of the proximal shaft 58. In the measuring apparatus 50, the proximal shaft 58 is pushed in from the second push-pull force gauge 54 side toward the distal side, whereby the outer tube 26 (the outer tube model M) is pressed onto the first push-pull force gauge 52 side. Based on measurement results of a pushing-in load exerted by the second push-pull force gauge 54 on the proximal side and a load measured at the first push-pull force gauge 52 on the distal side, the coefficient of transmission of a pushing-in load from the proximal side to the distal side is measured.

As shown in FIG. 5, in this experiment, a coefficient of transmission of load was measured in the condition where the inner tube 24 and the guide wire 20 are provided inside the outer tube model M, in other words, measured for the shaft main body 12, in order to realize a measurement condition close to the actual use condition of the catheter 10.

As shown in FIG. 6, the results of the experiment conducted using the measuring apparatus 50 showed that under a comparatively weak pushing-in load of 50 gf, a high load transmission coefficient of about 0.64 was obtained in substantially the same manner when the opening 22 was disposed at any of the points P1 to P4, and a low load transmission coefficient resulted only when the opening 22 was disposed at the point P5. Under strong pushing-in loads of 120 gf and 180 gf, on the other hand, a comparatively high load transmission coefficient was obtainable only in the condition where the opening 22 was disposed at the point P1 or the point P2.

The pushing-in load in an ordinary surgical procedure is supposed to amount to around 120 gf, in strong-push cases. Therefore, in order to configure a catheter 10 with an excellent load transmission performance, and taking into account the measurement results at pushing-in loads of 120 gf or more, it was concluded to be effective to provide the opening 22 at either of the positions P1 and P2, in other words, to provide the opening 22 in the second region R2. The first region R1 and the transition region R0 are devoid of any openings in the side wall.

Accordingly, in the catheter 10 in the present embodiment, the opening 22 is provided in the highly rigid second region R2, as shown in FIGS. 1, 2A and 2B. The distance from the proximal end of the transition region R0 to the center of the opening 22 is preferably, for example, about 5 to 40 mm, and the distance from the distal end of the outer tube 26 to the distal end of the opening 22 is preferably, for example, about 150 to 1,500 mm. These distance values may be optimized, if necessary, according to the specifications and use of the catheter 10.

The balloon 14 provided at the distal end of the catheter 10 is configured to be folded (deflated) and expanded (inflated) by variations in the internal pressure. As shown in FIG. 2B, the balloon 14 includes a tubular section (straight section) 14a capable of being expanded into a tubular shape (hollow cylindrical shape) by an expanding (inflating) fluid injected thereinto through the expansion lumen 26a, a distal tapered section 14b gradually decreasing in diameter on the distal side of the tubular section 14a, and a proximal tapered section 14c gradually decreasing in diameter on the proximal side of the tubular section 14a.

The balloon 14 is firmly attached to the shaft main body 12 by a structure in which a hollow cylindrical distal-side non-expansion part 14d provided on the distal side of the distal tapered section 14b is joined to the outer circumferential surface of the inner tube 24 in a liquid-tight manner, whereas a hollow cylindrical proximal-side non-expansion part 14e provided on the proximal side of the proximal tapered section 14c is joined to a distal portion of the outer tube 26 in a liquid-tight manner. The inside diameter of the distal-side non-expansion part 14d is approximately equal to the outside diameter of the inner tube 24, while the outside diameter of the proximal-side non-expansion part 14a is approximately equal to the inside diameter of the outer tube 26. It suffices for the balloon 14 and the inner and outer tubes 24, 26 to be firmly attached to each other in a liquid-tight manner; for example, the joining may be conducted by adhesion or heat fusing (welding).

The balloon 14, when expanded, is sized, for example, as follows. The tubular section 14a has an outside diameter of about 1 to 6 mm, preferably about 1 to 4 mm, and a length of about 5 to 50 mm, preferably about 5 to 40 mm. In addition, the distal-side non-expansion part 14d has an outside diameter of about 0.5 to 1.5 mm, preferably about 0.6 to 1.3 mm, and a length of about 1 to 5 mm, preferably about 1 to 2 mm. The proximal-side non-expansion part 14e has an outside diameter of about 0.5 to 1.6 mm, preferably about 0.7 to 1.5 mm, and a length of about 1 to 5 mm, preferably about 2 to 4 mm. Furthermore, the distal tapered section 14b and the proximal tapered section 14c each have a length of about 1 to 10 mm, preferably about 3 to 7 mm.

The balloon 14 as above is required to have an appropriate degree of flexibility, like the inner tube 24 and the outer tube 26, and is required to have such an extent of strength as to be able to securely push open a stenosed part. Thus, the material for the balloon 14 may be any of the above-mentioned materials for the inner tube 24 and the outer tube 26; naturally, other materials can also be used.

The operation of the catheter 10 according to the present embodiment which is configured as above will be described below.

First, the form of the stenosed part (lesion part) generated in a coronary artery or the like is determined by an intravascular imaging method or intravascular ultrasound diagnosis. Next, a guide wire 20 is precedently led into a blood vessel in a percutaneous manner from a femoral region or the like by the Seldinger catheter technique, for example. In addition, the guide wire 20 is passed through the wire lumen 24a, with the distal-side opening 24b of the inner tube 24 as an entrance, and, while leading out the guide wire 20 to the opening 22, the catheter 10 is inserted into the coronary artery. Then, under radiography, the guide wire 20 is advanced to the target stenosed part, is passed through the stenosed part and put indwelling there, and the catheter 10 is advanced along the guide wire 20 into the coronary artery. As a result, the distal end of the catheter 10 reaches the stenosed part, and is passed through (is made to penetrate) the stenosed part. This makes it possible to dispose the balloon 14 in the stenosed part. By feeding the expanding fluid (for example, a radiopaque material) under pressure from the hub 18 side into the expansion lumens 27a and 26a, therefore, the balloon 14 can be expanded (inflated) to dilate the stenosed part, thereby achieving a prescribed treatment.

In this case, the catheter 10 in this embodiment has a configuration (rapid exchange type) in which the opening 22 is provided at an intermediate portion of the shaft main body 12. Therefore, the catheter 10 may be shorter than in the case of a configuration (over-the-wire type) in which the guide wire 20 is led out to the proximal side of the hub 18. Accordingly, the catheter 10 is easier to handle, and the catheter 10 can be relatively easily exchanged in the condition where the guide wire 20 is set indwelling in the living body.

In addition, the outer tube 26 has the flexible first region R1, the highly rigid second region R2, and the transition region R0 varying in rigidity so as to interconnect the first and second regions R1, R2. This structure enables a configuration in which shaft rigidity is gradually lowered from the proximal side toward the distal side. Consequently, the catheter 10 can be smoothly advanced through a bent blood vessel or into a stenosed part having a rugged shape.

Moreover, since the opening 22 is disposed in the second region R2 provided as a highly rigid region, the coefficient of transmission of the pushing-in force from the proximal side to the distal side of the catheter 10 can be maintained at a high value (see FIG. 6), and an intuitive and stable feeling of operation can be obtained. In particular when the distal end of the catheter 10 is to penetrate a relatively hard stenosed part or the like, a sufficient pushing-in load can be transmitted to the distal end. Specifically, the pushing-in force exerted by the operator from the proximal side is first transmitted to the second region R2, which is high in rigidity; since the opening 22 is formed in this highly rigid second region R2, the absorption of the pushing-in force at the opening 22 part is minimized. Subsequently, therefore, the pushing-in force is appropriately transmitted to the transition region R0, where rigidity varies, and then to the flexible first region R1.

As for the outer tube 26, it is also effective to integrally mold the first region R1, the second region R2 and the transition region R0 by the above-mentioned manufacturing apparatus 30 or the like. This helps ensure that no joint part is formed at any intermediate portion of the outer tube 26, and, moreover, the rigidity of the outer tube 26 can be varied further smoothly. Therefore, the outer tube 26 is free of a region where rigidity varies abruptly, and it is possible to obviate a situation in which the joint part or the opening 22 part might constitute a rigidity change point such as to be a starting point of kinking or breakage under a tensile or bending load. In other words, the configuration wherein the outer tube 26 as a single tube is provided with the opening 22 in its highly rigid second region R2 and wherein the inner tube 24 is inserted via the opening 22 and joined to the outer tube 26 by heat fusing (welding) or the like, makes it possible to configure a catheter 10 having a shaft main body 12 which is higher in load transmission performance and higher in strength against loads such as a tensile load or a bending load. Moreover, where the outer tube 26 is formed as a single tube, the shaft main body 12 can be made relatively small in outside diameter over the whole part thereof, particularly in the vicinity of the opening 22. In addition, there is no need for a step of interconnecting a plurality of tubes, so that the manufacturing cost of the catheter can be cut down.

The invention is not restricted to the above-mentioned embodiment, and, naturally, various configurations or steps can be adopted within the scope of the invention.

For instance, the catheter 10 may not necessarily have the configuration in which the outer tube 26 is an integrally molded tube; instead, a configuration may be adopted in which tubes differing in rigidity and corresponding respectively to the first region R1 and the second region R2 are joined respectively to the distal end and the proximal end of a tube which varies in rigidity like the transition region R0. In this case, when the catheter 10 is operated with a very strong pushing-in force, there may arise a fear of kinking or the like, since rigidity varies somewhat sharply at each joint part between the tubes. With the opening 22 disposed in the second region R2 where rigidity is the highest in the outer tube 26, however, it is ensured that the pushing-in force transmission performance is rarely lowered at any part of the shaft main body 12, so that such a configuration can be used sufficiently effectively, depending on the use conditions for the catheter 10.

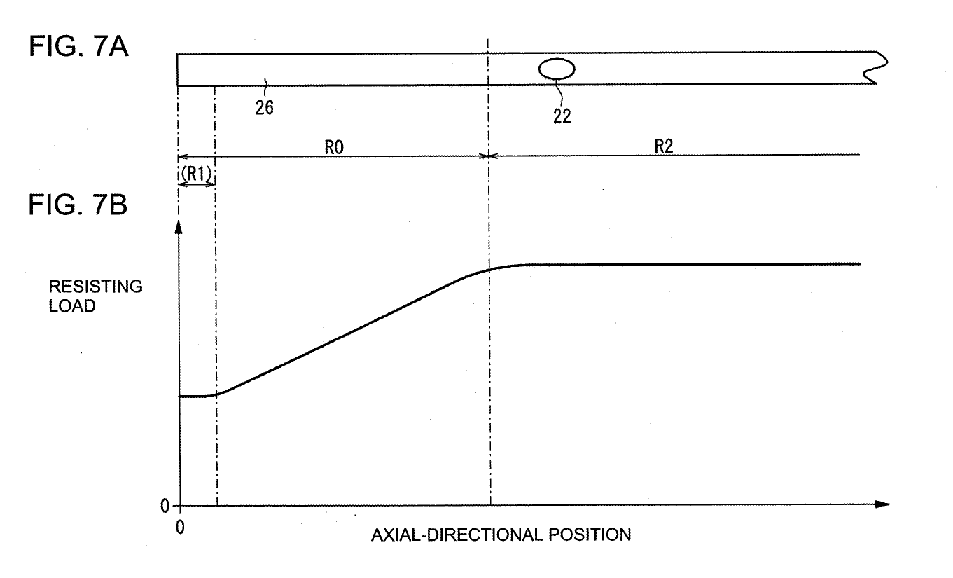

In the above description, a tube with a three-region structure including the first region R1, the second region R2 and the transition region R0 has been described as an example of the outer tube 26. This, however, is not restrictive of the invention. For example, a tube with a configuration in which the first region R1 and the second region R2 are integrally molded while the first region R1 is minimized in length or substantially omitted, as shown in FIGS. 7A and 7B, may be adopted as the outer tube 26. Naturally, the catheter 10 is not restricted to the three-region configuration including the first region R1, the second region R2 and the transition region R0, but may have a two-region configuration or a configuration having four or more regions. In such a case, also, a good load transmission coefficient can be obtained, by forming the opening 22 in a region which is the highest of all the regions in rigidity or in a region where rigidity is high to a certain extent.

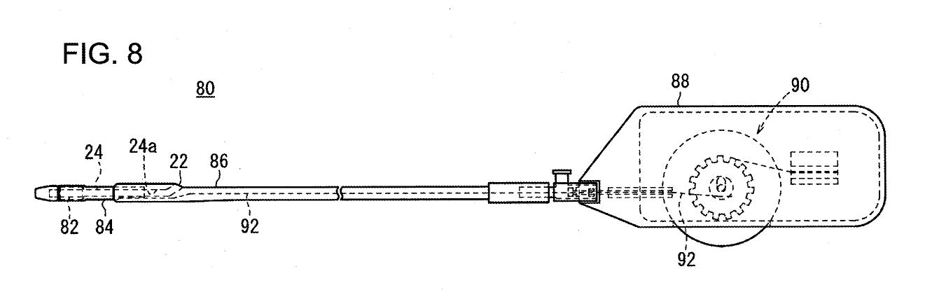

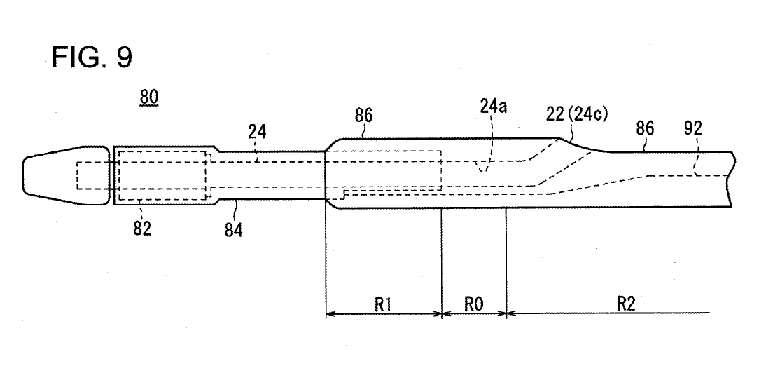

In addition, instead of providing the balloon 14 at the distal portion of the catheter 10, a catheter 80 as shown in FIGS. 8 and 9 may be configured which is applicable as the above-mentioned self-expandable stent catheter, for example.

Such a catheter 80 can be configured in substantially the same manner as the biorgan-dilating instrument described in Japanese Patent Laid-open No. 2006-305335, for example. Specifically, the catheter 80 includes an inner tube 24 formed therein with a wire lumen 24a in which a guide wire is to be passed, a stent-containing tube 84 for containing a stent 82 which is disposed on the distal side of the inner tube 24, and an outer tube 86 into a distal portion of which a proximal portion of the stent-containing tube 84 is to be inserted.

The stent-containing tube 84 can be withdrawn by a traction wire 92 which can be taken up by a take-up mechanism 90 mounted on an operating unit 88 provided on the proximal side of the outer tube 86, whereby the stent 82 can be opened in a living body. With such a catheter 80, also, a catheter having a good load transmission coefficient can be configured, by providing the outer tube 86 with a first region R1 and a second region R2 (and a transition region R0) and forming an opening 22 in the second region R2 where rigidity is relatively high.

The detailed description above describes features and aspects of examples of embodiments of a catheter. The present invention is not limited, however, to the precise embodiment and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A catheter comprising:
an outer tube possessing a distal end portion terminating at a distal-most end and a proximal end portion terminating at a proximal-most end, the outer tube including a tube wall surrounding an interior of the outer tube;
an inner tube disposed within the interior of the outer tube so that the outer tube surrounds and axially overlaps a proximal portion of the inner tube, the inner tube possessing a distal end extending distally beyond the distal-most end of the outer tube, the inner tube including a wire lumen extending along a longitudinal extent of the inner tube between a distal-end opening at the distal end of the inner tube and a proximal-end opening at a proximal end of the inner tube, the wire lumen being configured to receive a guide wire passing through both the distal-end opening and the proximal-end opening;
the outer tube including a through opening passing through the wall of the outer tube and opening to the interior of the outer tube, the opening being located distal of the proximal-most end of the outer tube, the proximal-most end of the inner tube being fixed in the opening;
the outer tube including a first region, a second region and a transition region arranged along an axial extent of the outer tube, the transition region being located axially between the first region and the second region, the first region being positioned distally of the transition region and the second region being positioned proximally of the transition region;
the second region possessing a rigidity greater than a rigidity of the first region;
the transition region between the first region and the second region possessing a rigidity which gradually varies from a distal end of the transition region which possesses the same rigidity as the rigidity of the first region to a proximal end of the transition region which possesses the same rigidity as the rigidity of the second region; and
the opening being located in the second region of the outer tube whose rigidity is greater than the rigidity of the first region.

2. The catheter according to claim 1, wherein the first region and the second region are formed respectively from resins differing in rigidity, and the transition region is so formed that a ratio of the resin of the first region and the resin of the second region varies along the axial extent of the transition region.

3. The catheter according to claim 2, wherein the first region, the second region, and the transition region are integrally molded in one piece.

4. The catheter according to claim 1, further comprising a balloon possessing a proximal end fixed to a distal end of the outer tube and possessing a distal end attached to a distal end of the inner tube.

5. The catheter according to claim 4, wherein the distal-most end of the inner tube extends distally beyond a distal-most end of the balloon.

6. The catheter according to claim 1, further comprising a proximal shaft possessing a distal end portion disposed within the interior of the outer tube, the proximal shaft possessing a lumen communicating with an open distal end of the proximal shaft.

7. The catheter according to claim 6, further comprising an expansion lumen between an inner surface of the outer tube and an outer surface of the inner tube, the lumen in the proximal shaft communicating with the expansion lumen, and the expansion lumen communicating with an interior of the balloon.

8. The catheter according to claim 1, wherein the first region of the outer tube is comprised only of a first resin composition, the second region of the outer tube is comprised of only a second resin composition different from the first resin composition, and the transition region is comprised of a mixture of the first and second resin compositions.

9. The catheter according to claim 8, wherein the transition region includes a proximal half and a distal half, the proximal half of the transition region being located between the distal half of the transition region of the outer tube and the second region of the outer tube, the distal half of the transition region being located between the proximal half of the transition region of the outer tube and the second region of the outer tube, the proximal half of the transition region possessing a ratio of the second resin composition to the first resin composition that is not less than one, the distal half of the transition region possessing a ratio of the first resin composition to the second resin composition that is not less than one.

10. A catheter comprising:
an outer tube;
an inner tube disposed within the outer tube and through which a guide wire is passed via a distal-side opening of the inner tube and a proximal-side opening of the inner tube;
the outer tube includes, in an axial direction of the outer tube, at least a first region on a distal side, a second region which is on a proximal side and which possesses a rigidity greater than the first region, and a transition region between the first region and the second region and which possesses a rigidity varying from the same rigidity as the rigidity of the first region to the same rigidity as the rigidity of the second region; and
the second region of the outer tube is provided with an opening to which the proximal-side opening of the inner tube is connected.

11. The catheter according to claim 10, wherein the first region and the second region are formed respectively from resins different in rigidity, and the transition region is so formed that a mixing ratio of the resin of the first region and the resin of the second region varies in an axial direction thereof.

12. The catheter according to claim 11, wherein the first region, the second region, and the transition region are integrally molded in one piece by extrusion using a resin change-over die.

13. The catheter according to claim 11, further comprising a balloon possessing a proximal end attached to a distal end of the outer tube and possessing a distal end attached to a distal end of the inner tube.

14. The catheter according to claim 13, wherein the inner tube possesses a distal-most end extending distally beyond a distal-most end of the balloon.

15. The catheter according to claim 11, further comprising a proximal shaft possessing a distal end portion disposed within the interior of the outer tube, the proximal shaft possessing a lumen communicating with an open distal end of the proximal shaft.

16. The catheter according to claim 15, further comprising an expansion lumen between an inner surface of the outer tube and an outer surface of the inner tube, the lumen in the proximal shaft communicating with the expansion lumen, and the expansion lumen communicating with an interior of the balloon.

17. The catheter according to claim 11, wherein the first region of the outer tube is comprised only of a first resin composition, the second region of the outer tube is comprised of only a second resin composition different from the first resin composition, and the transition region is comprised of a mixture of the first and second resin compositions.

18. The catheter according to claim 17, wherein the transition region includes a proximal half and a distal half, the proximal half of the transition region being located between the distal half of the transition region of the outer tube and the second region of the outer tube, the distal half of the transition region being located between the proximal half of the transition region of the outer tube and the second region of the outer tube, the proximal half of the transition region possessing a ratio of the second resin composition to the first resin composition that is not less than one, the distal half of the transition region possessing a ratio of the first resin composition to the second resin composition that is not less than one.
